(19) United States
(12) Patent Application Publication
(10) Pub. No.: US 2021/0047595 A1
(43) Pub. Date: Feb. 18, 2021

(54) CELL CULTURE DEVICE, AND CELL CULTURE METHOD USING SAME

(71) Applicant: UBE INDUSTRIES, LTD., Ube-shi, Yamaguchi (JP)

(72) Inventors: Masahiko HAGIHARA, Ube-shi (JP); Takashi HARADA, Ube-shi (JP); Akihiro MATSUBAYASHI, Ichihara-shi (JP); Shinsaku FUSE, Ube-shi (JP)

(21) Appl. No.: 16/963,855

(22) PCT Filed: Jan. 24, 2019
(86) PCT No.: PCT/JP2019/002377

§ 371 Date: Jul. 22, 2020

(30) Foreign Application Priority Data
Jan. 24, 2018 (JP) 2018-010108

(51) Int. Cl. C12M 3/00; C12M 1/12; C12M 1/10

(52) U.S. Cl. CPC C12M 3/00; B01F 7/00008; C12M 1/10; C12M 25/02

(57) ABSTRACT

The present invention provides a cell culture device characterized by comprising: a culture container; a rotating polymeric porous film housing part which is housed in the culture container, and has one or more culture medium inlet/outlet openings; and a polymeric porous film which is housed in the rotating polymeric porous film housing part, wherein the polymeric porous film is a modular polymeric porous film, and the rotating polymeric porous film housing part is rotated independently of the culture container.

### FIELD

The present invention relates to a cell culture device including a porous polymer film. In addition, the present invention relates to a cell culture method using the cell culture device including a porous polymer film.

### BACKGROUND

In recent years, proteins such as enzymes, hormones, antibodies, cytokines, viruses (viral proteins) used for treatment and vaccine are industrially produced using cultured cells. However, technologies to produce such proteins have problems in view of efficiency, exerting influence on the timely and stable supply of biomedicines of which the continuous and broad supply is necessary. Therefore, technologies to densely culture cells, and innovative and easy technologies to increase the amount of produced protein, such as continuous production methods with high efficiency have been demanded for establishing efficient, stable, and quick methods of producing proteins.

As cells for protein production, anchorage-dependent adherent cells which adhere to a culture substrate may be sometimes used. Since such cells grow anchorage-dependently, they need to be cultured while being adhered onto the surface of a petri dish, plate or chamber. Conventionally, in order to culture such adherent cells in a large amount, it was preferable to increase the surface area to be adhered. However, increasing the culturing area inevitably requires to increase the space, which is responsible for impairing efficient production and improvement in production amount.

As a method to culture a large amount of adherent cells while decreasing the culture space, a method for culture using a microporous carrier, especially a microcarrier, has been developed (for example, PTL 1). In a cell culturing system using microcarriers, it is preferable to carry out sufficient stirring and diffusion so that the microcarriers do not aggregate together. Since this requires a volume allowing sufficient stirring and diffusion of the medium in which the microcarriers are dispersed, there is an upper limit to the density at which the cells can be cultured. In order to separate the microcarrier from the medium, separation is preferably performed using a filter which can separate fine particles, possibly resulting in the deterioration of the productivity of biomedicines. Considering the foregoing, there is a demand for innovative methodology for cell culture which stably and easily cultures a large amount of adhered cells.

<Porous Polyimide Film>

Porous polyimide films have been utilized in the prior art for filters and low permittivity films, and especially for battery-related purposes, such as fuel cell electrolyte membrane and the like. PTLs 2 to 4 describe porous polyimide films with numerous macrovoids, having excellent permeability to objects such as gases, high porosity, excellent smoothness on both surfaces, relatively high strength and, despite high porosity, excellent resistance against compression stress in the film thickness direction. All of these are porous polyimide films formed via amic acid.

The cell culture method which includes applying cells to a porous polyimide film and culturing them is reported (PTL 5).

### CITATION LIST

### Patent Literature

[PTL 1] WO2003/054174
[PTL 2] WO2010/038873
[PTL 3] Japanese Unexamined Patent Publication (Kokai) No. 2011-219585
[PTL 4] Japanese Unexamined Patent Publication (Kokai) No. 2011-219586
[PTL 5] WO2015/012415

### SUMMARY

### Technical Problem

In order to culture a large amount of animal cells, it is preferable to continuously dissolve a large amount of oxygen in a medium. Examples of methods of dissolving oxygen in a medium include methods of stirring a medium, and bubbling methods. However, most of animal cells are vulnerable to a shearing force, and have a problem that the cells are killed by a stirring culture method. In a stirring culture method in combination of a usual magnetic stirrer and a stirring bar, cells are crushed in a contact between a culture vessel and the stirring bar, and it is difficult to culture a large amount of cells. In addition, a culture method with bubbling has a problem that cells are killed by bubbles generated at the time of the bubbling.

### Solution to Problem

The present inventors found that the porous polymer film having a prescribed structure not only provides an optimal space in which a large amount of cells can be cultured but also is capable of culturing a large amount of cells under a stirring condition under which a shearing force is generated, or under a culture condition under which bubbles are generated, and the present invention was thus accomplished. In other words, the present invention includes, but is not limited to, the following modes.

[1] A cell culture device comprising:

a culture vessel;

a rotary porous polymer film container that is contained in the culture vessel and has one or more medium flow inlets; and

a porous polymer film contained in the rotary porous polymer film container;

wherein the porous polymer film is a porous polymer film with a three-layer structure, comprising a surface layer A and a surface layer B having a plurality of pores, as well as a macrovoid layer sandwiched between the surface layer A and the surface layer B; an average pore diameter of the pores present in the surface layer A is smaller than an average pore diameter of the pores present in the surface layer B; the macrovoid layer comprises a partition wall bonded to surface layers A and B, and a plurality of macrovoids surrounded by the partition wall and the surface layers A and B; and the pores in the surface layers A and B communicate with the macrovoids;

wherein the porous polymer film is a modularized porous polymer film; and

wherein the rotary porous polymer film container is rotated independently of the culture vessel.

[2] The cell culture device according to [1], further comprising rotational driving means for rotating the rotary porous polymer film container.
[3] The cell culture device according to [2], wherein the rotary porous polymer film container comprises rotative power reception means.
[4] The cell culture device according to [3], wherein the rotative power reception means is a magnet.
[5] The cell culture device according to [4], wherein the rotational driving means is a magnetic stirrer.
[6] The cell culture device according to any one of [1] to [5],

wherein the modularized porous polymer film is a modularized porous polymer film comprising a casing; and

wherein in the modularized porous polymer film,

(i) the two or more independent porous polymer films being aggregated,

(ii) the porous polymer films being folded up,

(iii) the porous polymer films being wound into a roll-like shape, and/or

(iv) the porous polymer films being tied into a rope-like shape are contained in the casing.

[7] The cell culture device according to any one of [1] to [6], wherein the porous polymer film has a plurality of pores having an average pore diameter of 0.01 to 100 μm.
[8] The cell culture device according to any one of [1] to [7], wherein an average pore diameter of the surface layer A is 0.01 to 50 μm.
[9] The cell culture device according to any one of [1] to [8], wherein an average pore diameter of the surface layer B is 20 to 100 μm.
[10] The cell culture device according to any one of [1] to [9], wherein a total film thickness of the porous polymer film is 5 to 500 μm.
[11] The cell culture device according to any one of [1] to [10], wherein the porous polymer film is a porous polyimide film.
[12] The cell culture device according to [11], wherein the porous polyimide film is a porous polyimide film comprising a polyimide derived from tetracarboxylic dianhydride and diamine.
[13] The cell culture device according to [11] or [12], wherein the porous polyimide film is a colored porous polyimide film that is obtained by molding a polyamic acid solution composition comprising a polyamic acid solution derived from tetracarboxylic dianhydride and diamine, and a coloring precursor, and subsequently heat-treating the resultant composition at 250° C. or more.
[14] The cell culture device according to any one of [1] to [10], wherein the porous polymer film is a porous polyethersulfone (PES) film.
[15] A method of culturing cells, using the cell culture device according to any one of [1] to [14].

### Advantageous Effects of Invention

The present invention is to provide: a new cell culture device in which culture is possible even under a stirring condition under which a shearing force is generated, cells are prevented from being crushed, and cells are not killed by bubbles; and a culture method using the cell culture device.

### BRIEF DESCRIPTION OF DRAWINGS

FIG. 1 is a cross-sectional view illustrating a cell culture device according to one embodiment.

FIG. 2 is a perspective view illustrating a portion of a cell culture device according to one embodiment.

FIG. 3 is a plan view illustrating a portion of a cell culture device according to one embodiment.

FIG. 4 is a cross-sectional view illustrating a portion of a cell culture device according to one embodiment, and illustrates a cross section taken along the line A-A of FIG. 3(A).

FIG. 5 is a plan view illustrating a modularized porous polyimide film applied to a cell culture device in accordance with one embodiment.

FIG. 6 is a cross-sectional view illustrating a modularized porous polyimide film applied to a cell culture device in accordance with one embodiment, and illustrates a cross section taken along the line B-B of FIG. 5.

FIG. 7 illustrates a portion of a cell culture device according to one embodiment. (A) is a side view, (B) is a perspective view, and (C) is a perspective view (without an apex part).

FIG. 8 illustrates a portion of a cell culture device according to one embodiment, and is a diagram obtained by cutting each of FIGS. 7(A) to 7(C) along a plane passing through the center of a rotation axis.

FIG. 9 is a photograph illustrating a mode in which a cell culture device is used in accordance with one embodiment. (A) represents the cell culture device placed in an incubator, (B) represents a rotary porous polymer film container (without an apex part) in which a modularized porous polymer film is placed, and (C) represents the rotary porous polymer film container (with the apex part) in which the modularized porous polymer film is placed.

FIG. 10 represents glucose concentration (GLC) and lactic acid concentrations (LAC) after culture using a cell culture device of one embodiment.

### DESCRIPTION OF EMBODIMENTS

Embodiments of the present invention will be described below with reference to the drawings as needed. The configurations of the embodiments are illustrative, and the constitution of the present invention is not limited to the specific configurations of the embodiments.

1. Porous Polymer Film

An average pore diameter of the pore present on a surface layer A (hereinafter referred to as “surface A” or “mesh surface”) in the porous polymer film used for the present invention is not particularly limited, but is, for example, 0.01 μm or more and less than 200 μm, 0.01 to 150 μm, 0.01 to 100 μm, 0.01 to 50 μm, 0.01 to 40 μm, 0.01 to 30 μm, 0.01 to 25 μm, 0.01 to 20 μm, or 0.01 to 15 μm, preferably 0.01 to 25 μm.

The average pore diameter of the pore present on a surface layer B (hereinafter referred to as “surface B” or “large pore surface”) in the porous polymer film used for the present invention is not particularly limited so long as it is larger than the average pore diameter of the pore present on the surface A, but is, for example, greater than 5 μm and 200 μm or less, 20 μm to 100 μm, 25 μm to 100 μm, 30 μm to 100 μm, 35 μm to 100 μm, 40 μm to 100 μm, 50 μm to 100 μm, or 60 μm to 100 μm, preferably 30 μm to 100 μm.

In this specification, the average pore diameter on the surface of the porous polymer film is an area-averaged pore diameter. The area-averaged pore diameter can be determined according to the following (1) and (2). The average pore diameter of a site other than the surface of the porous polymer film can also be determined in a similar manner.

(1) Pore areas S for 200 or more open pore portions are measured from a scanning electron micrograph of the surface of the porous film, and each pore diameter d is determined according to Equation I assuming that the pore areas have a perfect circle.

[Math. 1]

$$\begin{matrix}[ Math . 1 ] & \\ \begin{matrix}Pore \\ Diameter d\end{matrix} = 2 \times \sqrt{( S \text{/} π )} & Equation I\end{matrix}$$

(2) All the pore diameters demanded by the above Equation I are applied to the following Equation II, and an area-averaged pore diameter da is determined assuming that the shape of each pore is a perfect circle.

[Math. 2]

$$\begin{matrix}[ Math . 2 ] & \\ \begin{matrix}Area \text{-} Average Pore \\ Diameter d a\end{matrix} = \sum ( {d}^{3} ) \text{/} \sum ( {d}^{2} ) & Equation II\end{matrix}$$

The thicknesses of the surface layers A and B are not particularly limited, but is, for example, 0.01 to 50 μm, preferably 0.01 to 20 μm.

The average pore diameter of macrovoids in the planar direction of the film in the macrovoid layer in the porous polymer film is not particularly limited but is, for example, 10 to 500 μm, preferably 10 to 100 μm, and more preferably 10 to 80 μm. The thicknesses of the partition wall in the macrovoid layer are not particularly limited, but is, for example, 0.01 to 50 μm, preferably 0.01 to 20 μm. In an embodiment, at least one partition wall in the macrovoid layer has one or two or more pores connecting the neighboring macrovoids and having the average pore diameter of 0.01 to 100 μm, preferably 0.01 to 50 μm. In another embodiment, the partition wall in the macrovoid layer has no pore.

The total film thickness of the porous polymer film used for the invention is not particularly limited, but may be 5 μm or more, 10 μm or more, 20 μm or more or 25 μm or more, and 500 μm or less, 300 μm or less, 100 μm or less, 75 μm or less, or 50 μm or less. It is preferably 5 to 500 μm, and more preferably 25 to 75 μm.

The film thickness of the porous polymer film used for the invention can be measured using a contact thickness gauge.

The porosity of the porous polymer film used in the present invention is not particularly limited but is, for example, 40% or more and less than 95%.

The porosity of the porous polymer film used for the invention can be determined by measuring the film thickness and mass of the porous film cut out to a prescribed size, and performing calculation from the basis weight according to the following Equation III.

[Math. 3]

Porosity (%)=(1−w/(S×d×D))× 100Equation III

(wherein S represents the area of the porous film, d represents the total film thickness, w represents the measured mass, and D represents the polymer density. The density is defined as 1.34 g/cm³ when the polymer is a polyimide.)

The porous polymer film used for the present invention is preferably a porous polymer film which includes a three-layer structure porous polymer film having a surface layer A and a surface layer B, the surface layers having a plurality of pores, and a macrovoid layer sandwiched between the surface layers A and B; wherein the average pore diameter of the pore present on the surface layer A is 0.01 μm to 25 μm, and the average pore diameter of the pore present on the surface layer B is 30 μm to 100 μm; wherein the macrovoid layer has a partition wall bonded to the surface layers A and B, and a plurality of macrovoids surrounded by such a partition wall and the surface layers A and B, the thickness of the macrovoid layer, and the surface layers A and B is 0.01 to 20 μm; wherein the pores on the surface layers A and B communicate with the macrovoid, the total film thickness is 5 to 500 μm, and the porosity is 40% or more and less than 95%. In an embodiment, at least one partition wall in the macrovoid layer has one or two or more pores connecting the neighboring macrovoids with each other and having the average pore diameter of 0.01 to 100 μm, preferably 0.01 to 50 μm. In another embodiment, the partition wall does not have such pores.

The porous polymer film used for the present invention is preferably sterilized. The sterilization treatment is not particularly limited, but any sterilization treatment such as dry heat sterilization, steam sterilization, sterilization with a disinfectant such as ethanol, electromagnetic wave sterilization such as ultraviolet rays or gamma rays, and the like can be mentioned.

The porous polymer film used for the present invention is not particularly limited so long as it has the structural features described above and includes, preferably a porous polyimide film or porous polyethersulfone (PES) film.

1-1. Porous Polyimide Film

Polyimide is a general term for polymers containing imide bonds in the repeating unit, and usually it refers to an aromatic polyimide in which aromatic compounds are directly linked by imide bonds. An aromatic polyimide has an aromatic-aromatic conjugated structure via an imide bond, and therefore has a strong rigid molecular structure, and since the imide bonds provide powerful intermolecular force, it has very high levels of thermal, mechanical and chemical properties.

The porous polyimide film usable for the present invention is a porous polyimide film preferably containing polyimide (as a main component) obtained from tetracarboxylic dianhydride and diamine, more preferably a porous polyimide film composed of tetracarboxylic dianhydride and diamine. The phrase “including as the main component” means that it essentially contains no components other than the polyimide obtained from a tetracarboxylic dianhydride and a diamine, as constituent components of the porous polyimide film, or that it may contain them but they are additional components that do not affect the properties of the polyimide obtained from the tetracarboxylic dianhydride and diamine.

In an embodiment, the porous polyimide film usable for the present invention includes a colored porous polyimide film obtained by forming a polyamic acid solution composition including a polyamic acid solution obtained from a tetracarboxylic acid component and a diamine component, and a coloring precursor, and then heat treating it at 250° C. or higher.

A polyamic acid is obtained by polymerization of a tetracarboxylic acid component and a diamine component. A polyamic acid is a polyimide precursor that can be cyclized to a polyimide by thermal imidization or chemical imidization.

The polyamic acid used may be any one that does not have an effect on the invention, even if a portion of the amic acid is imidized. Specifically, the polyamic acid may be partially thermally imidized or chemically imidized.

When the polyamic acid is to be thermally imidized, there may be added to the polyamic acid solution, if necessary, an imidization catalyst, an organic phosphorus-containing compound, or fine particles such as inorganic fine particles or organic fine particles. In addition, when the polyamic acid is to be chemically imidized, there may be added to the polyamic acid solution, if necessary, a chemical imidization agent, a dehydrating agent, or fine particles such as inorganic fine particles or organic fine particles. Even if such components are added to the polyamic acid solution, they are preferably added under conditions that do not cause precipitation of the coloring precursor.

In this specification, a “coloring precursor” is a precursor that generates a colored substance by partial or total carbonization under heat treatment at 250° C. or higher.

Coloring precursors usable for the production of the porous polyimide film are preferably uniformly dissolved or dispersed in a polyamic acid solution or polyimide solution and subjected to thermal decomposition by heat treatment at 250° C. or higher, preferably 260° C. or higher, even more preferably 280° C. or higher and more preferably 300° C. or higher, and preferably heat treatment in the presence of oxygen such as air, at 250° C., preferably 260° C. or higher, even more preferably 280° C. or higher and more preferably 300° C. or higher, for carbonization to produce a colored substance, more preferably producing a black colored substance, with carbon-based coloring precursors being most preferred.

The coloring precursor, when being heated, first appears as a carbonized compound, but compositionally it contains other elements in addition to carbon, and also includes layered structures, aromatic crosslinked structures and tetrahedron carbon-containing disordered structures.

Carbon-based coloring precursors are not particularly restricted, and for example, they include tar or pitch such as petroleum tar, petroleum pitch, coal tar and coal pitch, coke, polymers obtained from acrylonitrile-containing monomers, ferrocene compounds (ferrocene and ferrocene derivatives), and the like. Of these, polymers obtained from acrylonitrile-containing monomers and/or ferrocene compounds are preferred, with polyacrylonitrile being preferred as a polymer obtained from an acrylonitrile-containing monomer.

Moreover, in another embodiment, examples of the porous polyimide film which may be used for the preset invention also include a porous polyimide film which can be obtained by molding a polyamic acid solution derived from a tetracarboxylic acid component and a diamine component followed by heat treatment without using the coloring precursor.

The porous polyimide film produced without using the coloring precursor may be produced, for example, by casting a polyamic acid solution into a film, the polyamic acid solution being composed of 3 to 60% by mass of polyamic acid having an intrinsic viscosity number of 1.0 to 3.0 and 40 to 97% by mass of an organic polar solvent, immersing or contacting in a coagulating solvent containing water as an essential component, and imidating the porous film of the polyamic acid by heat treatment. In this method, the coagulating solvent containing water as an essential component may be water, or a mixed solution containing 5% by mass or more and less than 100% by mass of water and more than 0% by mass and 95% by mass or less of an organic polar solvent. Further, after the imidation, one surface of the resulting porous polyimide film may be subjected to plasma treatment.

The tetracarboxylic dianhydride which may be used for the production of the porous polyimide film may be any tetracarboxylic dianhydride, selected as appropriate according to the properties desired. Specific examples of tetracarboxylic dianhydrides include biphenyltetracarboxylic dianhydrides such as pyromellitic dianhydride, 3,3′,4,4′-biphenyltetracarboxylic dianhydride (s-BPDA) and 2,3,3′,4′-biphenyltetracarboxylic dianhydride (a-BPDA), oxydiphthalic dianhydride, diphenylsulfone-3,4,3′,4′-tetracarboxylic dianhydride, bis(3,4-dicarboxyphenyl)sulfide dianhydride, 2,2-bis(3,4-dicarboxyphenyl)-1,1,1,3,3,3-hexafluoropropane dianhydride, 2,3,3′,4′-benzophenonetetracarboxylic dianhydride, 3,3′,4,4′-benzophenonetetracarboxylic dianhydride, bis(3,4-dicarboxyphenyl)methane dianhydride, 2,2-bis(3,4-dicarboxyphenyl)propane dianhydride, p-phenylenebis(trimellitic acid monoester acid anhydride), p-biphenylenebis(trimellitic acid monoester acid anhydride), m-terphenyl-3,4,3′,4′-tetracarboxylic dianhydride, p-terphenyl-3,4,3′,4′-tetracarboxylic dianhydride, 1,3-bis(3,4-dicarboxyphenoxy)benzene dianhydride, 1,4-bis(3,4-dicarboxyphenoxy)benzene dianhydride, 1,4-bis(3,4-dicarboxyphenoxy)biphenyl dianhydride, 2,2-bis[(3,4-dicarboxyphenoxy)phenyl]propane dianhydride, 2,3,6,7-naphthalenetetracarboxylic dianhydride, 1,4,5,8-naphthalenetetracarboxylic dianhydride, 4,4′-(2,2-hexafluoroisopropylidene)diphthalic dianhydride, and the like. Also preferably used is an aromatic tetracarboxylic acid such as 2,3,3′,4′-diphenylsulfonetetracarboxylic acid. These may be used alone or in appropriate combinations of two or more.

Particularly preferred among these are at least one type of aromatic tetracarboxylic dianhydride selected from the group consisting of biphenyltetracarboxylic dianhydride and pyromellitic dianhydride. As a biphenyltetracarboxylic dianhydride there may be suitably used 3,3′,4,4′-biphenyltetracarboxylic dianhydride.

As diamine which may be used for the production of the porous polyimide film, any diamine may be used. Specific examples of diamines include the following.

1) Benzenediamines with one benzene nucleus, such as 1,4-diaminobenzene(paraphenylenediamine), 1,3-diaminobenzene, 2,4-diaminotoluene and 2,6-diaminotoluene;

2) diamines with two benzene nuclei, including diaminodiphenyl ethers such as 4,4′-diaminodiphenyl ether and 3,4′-diaminodiphenyl ether, and 4,4′-diaminodiphenylmethane, 3,3′-dimethyl-4,4′-diaminobiphenyl, 2,2′-dimethyl-4,4′-diaminobiphenyl, 2,2′-bis(trifluoromethyl)-4,4′-diaminobiphenyl, 3,3′-dimethyl-4,4′-diaminodiphenylmethane, 3,3′-dicarboxy-4,4′-diaminodiphenylmethane, 3,3′,5,5′-tetramethyl-4,4′-diaminodiphenylmethane, bis(4-aminophenyl)sulfide, 4,4′-diaminobenzanilide, 3,3′-dichlorobenzidine, 3,3′-dimethylbenzidine, 2,2′-dimethylbenzidine, 3,3′-dimethoxybenzidine, 2,2′-dimethoxybenzidine, 3,3′-diaminodiphenyl ether, 3,4′-diaminodiphenyl ether, 4,4′-diaminodiphenyl ether, 3,3′-diaminodiphenyl sulfide, 3,4′-diaminodiphenyl sulfide, 4,4′-diaminodiphenyl sulfide, 3,3′-diaminodiphenylsulfone, 3,4′-diaminodiphenylsulfone, 4,4′-diaminodiphenylsulfone, 3,3′-diaminobenzophenone, 3,3′-diamino-4,4′-dichlorobenzophenone, 3,3′-diamino-4,4′-dimethoxybenzophenone, 3,3′-diaminodiphenylmethane, 3,4′-diaminodiphenylmethane, 4,4′-diaminodiphenylmethane, 2,2-bis(3-aminophenyl)propane, 2,2-bis(4-aminophenyl)propane, 2,2-bis(3-aminophenyl)-1,1,1,3,3,3-hexafluoropropane, 2,2-bis(4-aminophenyl)-1,1,1,3,3,3-hexafluoropropane, 3,3′-diaminodiphenyl sulfoxide, 3,4′-diaminodiphenyl sulfoxide and 4,4′-diaminodiphenyl sulfoxide;

3) diamines with three benzene nuclei, including 1,3-bis(3-aminophenyl)benzene, 1,3-bis(4-aminophenyl)benzene, 1,4-bis(3-aminophenyl)benzene, 1,4-bis(4-aminophenyl)benzene, 1,3-bis(4-aminophenoxy)benzene, 1,4-bis(3-aminophenoxy)benzene, 1,4-bis(4-aminophenoxy)benzene, 1,3-bis(3-aminophenoxy)-4-trifluoromethylbenzene, 3,3′-diamino-4-(4-phenyl)phenoxybenzophenone, 3,3′-diamino-4,4′-di(4-phenylphenoxy)benzophenone, 1,3-bis(3-aminophenyl sulfide)benzene, 1,3-bis(4-aminophenyl sulfide)benzene, 1,4-bis(4-aminophenyl sulfide)benzene, 1,3-bis(3-aminophenylsulfone)benzene, 1,3-bis(4-aminophenylsulfone)benzene, 1,4-bis(4-aminophenylsulfone)benzene, 1,3-bis[2-(4-aminophenyl)isopropyl]benzene, 1,4-bis[2-(3-aminophenyl)isopropyl]benzene and 1,4-bis[2-(4-aminophenyl)isopropyl]benzene;

4) diamines with four benzene nuclei, including 3,3′-bis(3-aminophenoxy)biphenyl, 3,3′-bis(4-aminophenoxy)biphenyl, 4,4′-bis(3-aminophenoxy)biphenyl, 4,4′-bis(4-aminophenoxy)biphenyl, bis[3-(3-aminophenoxy)phenyl]ether, bis[3-(4-aminophenoxy)phenyl]ether, bis[4-(3-aminophenoxy)phenyl]ether, bis[4-(4-aminophenoxy)phenyl]ether, bis[3-(3-aminophenoxy)phenyl]ketone, bis[3-(4-aminophenoxy)phenyl]ketone, bis[4-(3-aminophenoxy)phenyl]ketone, bis[4-(4-aminophenoxy)phenyl]ketone, bis[3-(3-aminophenoxy)phenyl]sulfide, bis[3-(4-aminophenoxy)phenyl]sulfide, bis[4-(3-aminophenoxy)phenyl]sulfide, bis[4-(4-aminophenoxy)phenyl]sulfide, bis[3-(3-aminophenoxy)phenyl]sulfone, bis[3-(4-aminophenoxy)phenyl]sulfone, bis[4-(3-aminophenoxy)phenyl]sulfone, bis[4-(4-aminophenoxy)phenyl]sulfone, bis[3-(3-aminophenoxy)phenyl]methane, bis[3-(4-aminophenoxy)phenyl]methane, bis[4-(3-aminophenoxy)phenyl]methane, bis[4-(4-aminophenoxy)phenyl]methane, 2,2-bis[3-(3-aminophenoxy)phenyl]propane, 2,2-bis[3-(4-aminophenoxy)phenyl]propane, 2,2-bis[4-(3-aminophenoxy)phenyl]propane, 2,2-bis[4-(4-aminophenoxy)phenyl]propane, 2,2-bis[3-(3-aminophenoxy)phenyl]-1,1,1,3,3,3-hexafluoropropane, 2,2-bis[3-(4-aminophenoxy)phenyl]-1,1,1,3,3,3-hexafluoropropane, 2,2-bis[4-(3-aminophenoxy)phenyl]-1,1,1,3,3,3-hexafluoropropane and 2,2-bis[4-(4-aminophenoxy)phenyl]-1,1,1,3,3,3-hexafluoropropane.

These may be used alone or in mixtures of two or more. The diamine used may be appropriately selected according to the properties desired.

Preferred among these are aromatic diamine compounds, with 3,3′-diaminodiphenyl ether, 3,4′-diaminodiphenyl ether, 4,4′-diaminodiphenyl ether, paraphenylenediamine, 1,3-bis(3-aminophenyl)benzene, 1,3-bis(4-aminophenyl)benzene, 1,4-bis(3-aminophenyl)benzene, 1,4-bis(4-aminophenyl)benzene, 1,3-bis(4-aminophenoxy)benzene and 1,4-bis(3-aminophenoxy)benzene being preferred for use. Particularly preferred is at least one type of diamine selected from the group consisting of benzenediamines, diaminodiphenyl ethers and bis(aminophenoxy)phenyl.

From the viewpoint of heat resistance and dimensional stability under high temperature, the porous polyimide film which may be used for the invention is preferably formed from a polyimide obtained by combination of a tetracarboxylic dianhydride and a diamine, having a glass transition temperature of 240° C. or higher, or without a distinct transition point at 300° C. or higher.

From the viewpoint of heat resistance and dimensional stability under high temperature, the porous polyimide film which may be used for the invention is preferably a porous polyimide film comprising one of the following aromatic polyimides.

(i) An aromatic polyimide comprising at least one tetracarboxylic acid unit selected from the group consisting of biphenyltetracarboxylic acid units and pyromellitic acid units, and an aromatic diamine unit,

(ii) an aromatic polyimide comprising a tetracarboxylic acid unit and at least one type of aromatic diamine unit selected from the group consisting of benzenediamine units, diaminodiphenyl ether units and bis(aminophenoxy)phenyl units, and/or,

(iii) an aromatic polyimide comprising at least one type of tetracarboxylic acid unit selected from the group consisting of biphenyltetracarboxylic acid units and pyromellitic acid units, and at least one type of aromatic diamine unit selected from the group consisting of benzenediamine units, diaminodiphenyl ether units and bis(aminophenoxy)phenyl units.

The porous polyimide film used in the present invention is preferably a three-layer structure porous polyimide film having a surface layer A and a surface layer B, the surface layers having a plurality of pores, and a macrovoid layer sandwiched between the surface layers A and B; wherein an average pore diameter of the pores present in the surface layer A is 0.01 μm to 25 μm, and the mean pore diameter present on the surface layer B is 30 μm to 100 μm; wherein the macrovoid layer has a partition wall bonded to the surface layers A and B, and a plurality of macrovoids surrounded by such a partition wall and the surface layers A and B; wherein the thickness of the macrovoid layer, and the surface layers A and B is 0.01 to 20 μm, wherein the pores on the surface layers A and B communicate with the macrovoid, the total film thickness is 5 to 500 μm, and the porosity is 40% or more and less than 95%. In this case, at least one partition wall in the macrovoid layer has one or two or more pores connecting the neighboring macrovoids and having the average pore diameter of 0.01 to 100 μm, preferably 0.01 to 50 μm.

For example, porous polyimide films described in WO2010/038873, Japanese Unexamined Patent Publication (Kokai) No. 2011-219585 or Japanese Unexamined Patent Publication (Kokai) No. 2011-219586 may be used for the present invention.

1-2. Porous Polyethersulfone (PES) Film

The porous PES film which may be used for the present invention contains polyethersulfone and typically consists substantially of polyethersulfone. Polyethersulfone may be synthesized by the method known to those skilled in the art. For example, it may be produced by a method wherein a dihydric phenol, an alkaline metal compound and a dihalogenodiphenyl compound are subjected to polycondensation reaction in an organic polar solvent, a method wherein an alkaline metal di-salt of a dihydric phenol previously synthesized is subjected to polycondensation reaction dihalogenodiphenyl compound in an organic polar solvent or the like.

Examples of an alkaline metal compound include alkaline metal carbonate, alkaline metal hydroxide, alkaline metal hydride, alkaline metal alkoxide and the like. Particularly, sodium carbonate and potassium carbonate are preferred.

Examples of a dihydric phenol compound include hydroquinone, catechol, resorcin, 4,4′-biphenol, bis (hydroxyphenyl)alkanes (such as 2,2-bis(hydroxyphenyl)propane, and 2,2-bis(hydroxyphenyl)methane), dihydroxydiphenylsulfones, dihydroxydiphenyl ethers, or those mentioned above having at least one hydrogen on the benzene rings thereof substituted with a lower alkyl group such as a methyl group, an ethyl group, or a propyl group, or with a lower alkoxy group such as a methoxy group, or an ethoxy group. As the dihydric phenol compound, two or more of the aforementioned compounds may be mixed and used.

Polyethersulfone may be a commercially available product. Examples of a commercially available product include SUMIKAEXCEL 7600P, SUMIKAEXCEL 5900P (both manufactured by Sumitomo Chemical Company, Limited).

The logarithmic viscosity of the polyethersulfone is preferably 0.5 or more, more preferably 0.55 or more from the viewpoint of favorable formation of a macrovoid of the porous polyethersulfone membrane; and it is preferably 1.0 or less, more preferably 0.9 or less, further preferably 0.8 or less, particularly preferably 0.75 or less from the viewpoint of the easy production of a porous polyethersulfone film.

Further, from the viewpoints of heat resistance and dimensional stability under high temperature, it is preferred that the porous PES film or polyethersulfone as a raw material thereof has a glass transition temperature of 200° C. or higher, or that a distinct glass transition temperature is not observed.

The method for producing the porous PES film which may be used for the present invention is not particularly limited. For example, the film may be produced by a method including the following steps:

a step in which polyethersulfone solution containing 0.3 to 60% by mass of polyethersulfone having logarithmic viscosity of 0.5 to 1.0 and 40 to 99.7% by mass of an organic polar solvent is casted into a film, immersed in or contacted with a coagulating solvent containing a poor solvent or non-solvent of polyethersulfone to produce a coagulated film having pores; and

a step in which the coagulated film having pores obtained in the above-mentioned step is heat-treated for coarsening of the aforementioned pores to obtain a porous PES film; wherein the heat treatment includes the temperature of the coagulated film having the pores is raised higher than the glass transition temperature of the polyethersulfone, or up to 240° C. or higher.

The porous PES film which can be used in the present invention is preferably a porous PES film having a surface layer A, a surface layer B, and a macrovoid layer sandwiched between the surface layers A and B,

wherein the macrovoid layer has a partition wall bonded to the surface layers A and B, and a plurality of macrovoids surrounded by such a partition wall and the surface layers A and B, the macrovoids having the average pore diameter in the planar direction of the film of 10 to 500 μm;

wherein the thickness of the macrovoid layer is 0.1 to 50 μm,

each of the surface layers A and B has a thickness of 0.1 to 50 μm,

wherein one of the surface layers A and B has a plurality of pores having the average pore diameter of more than 5 μm and 200 μm or less, while the other has a plurality of pores having the average pore diameter of 0.01 μm or more and less than 200 μm,

wherein one of the surface layers A and B has a surface aperture ratio of 15% or more while other has a surface aperture ratio of 10% or more,

wherein the pores of the surface layers A and B communicate with the macrovoids,

wherein the porous PES film has total film thickness of 5 to 500 μm and a porosity of 50 to 95%.

Since the above-described porous polymer film as a cell culture carrier, used in the cell culture device of the present invention, has a porous characteristic of slight hydrophilicity, stable liquid holding is achieved in the porous polymer film, and a wet environment even resistant to drying is maintained. Therefore, the survival and growth of cells can be achieved even with a very small amount of medium in comparison with a cell culture device using a conventional cell culture carrier. Since cells seeded on the porous polymer film can be cultured without killing cells even by a shearing force or bubbles, it is possible to efficiently supply oxygen and a nutrient to cells, and to culture a large amount of cells.

In accordance with the present invention, it is possible to sufficiently supply oxygen to cells.

2. Cell Culture Device

The present invention relates to a cell culture device comprising:

a culture vessel;

a rotary porous polymer film container that is contained in the culture vessel and has one or more medium flow inlets; and

a porous polymer film contained in the rotary porous polymer film container;

wherein the porous polymer film is a porous polymer film with a three-layer structure, comprising a surface layer A and a surface layer B having a plurality of pores, as well as a macrovoid layer sandwiched between the surface layer A and the surface layer B; an average pore diameter of the pores present in the surface layer A is smaller than an average pore diameter of the pores present in the surface layer B; the macrovoid layer comprises a partition wall bonded to surface layers A and B, and a plurality of macrovoids surrounded by the partition wall and the surface layers A and B; and the pores in the surface layers A and B communicate with the macrovoids;

wherein the porous polymer film is a modularized porous polymer film; and

wherein the rotary porous polymer film container is rotated independently of the culture vessel.

The cell culture device will be hereinafter referred to as a “cell culture device of the present invention”. Embodiments of the cell culture device of the present invention will be described with reference to the drawings.

2-1. Cell Culture Device

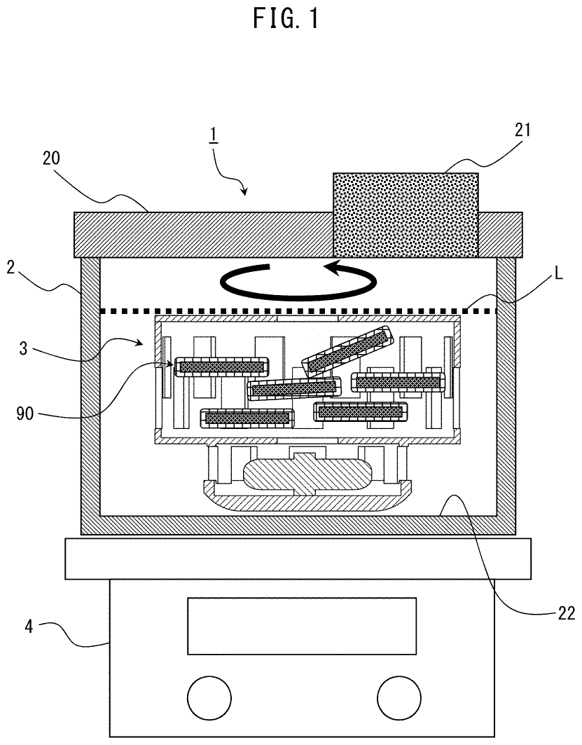

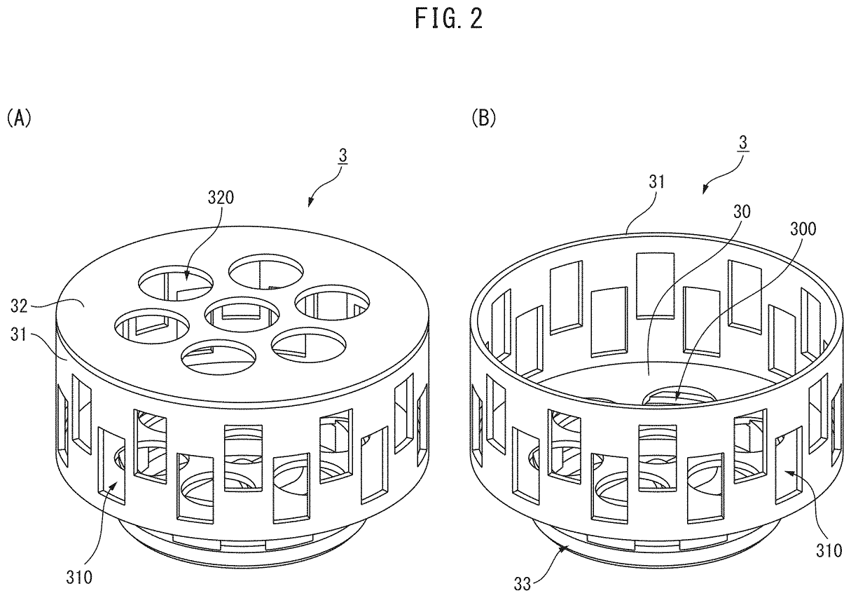

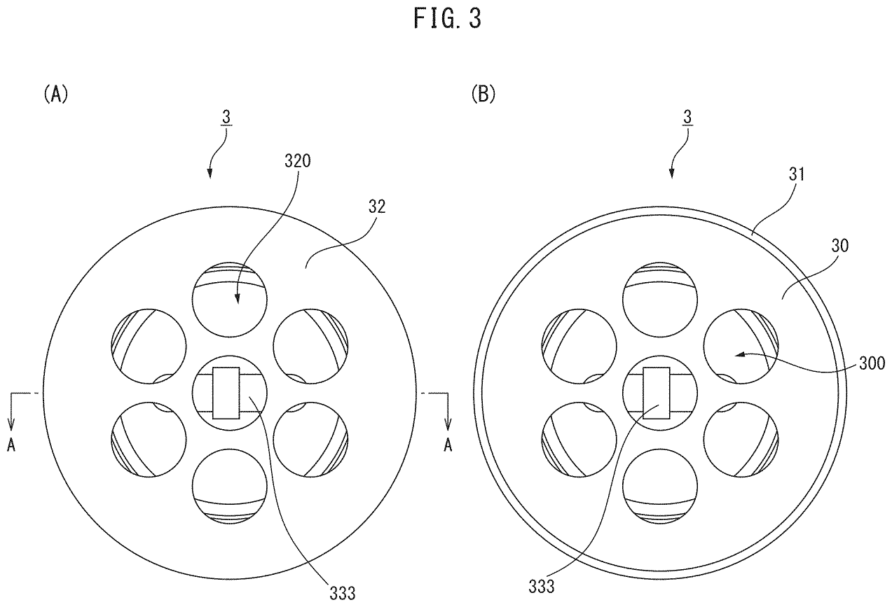

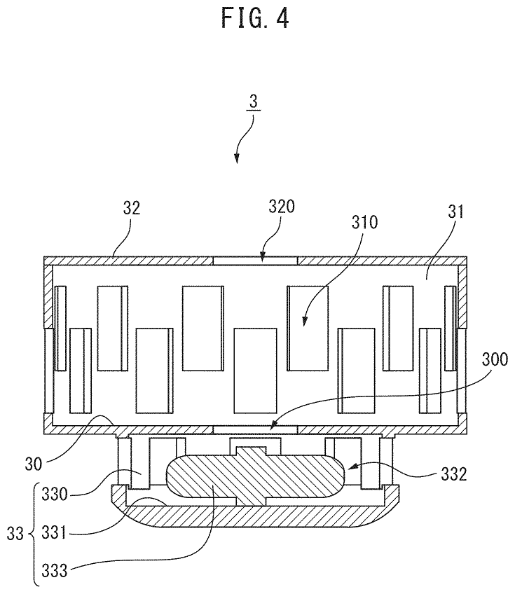

FIG. 1 is a diagram illustrating a cell culture device 1 of the present invention according to one embodiment. FIGS. 2 to 4 are diagrams illustrating a rotary porous polymer film container 3 according to one embodiment.

The rotary porous polymer film container 3 is contained in a culture vessel 2. In one embodiment, a lid 20 is further included for covering the culture space of the culture vessel 2. A part of the lid 20 preferably includes a filter 21 so that gas containing oxygen is supplied into the interior of the culture vessel 2. As a result, gas exchange between the interior and exterior of the culture vessel 2 is enabled, and a medium is prevented from being contaminated.

A modularized porous polymer film 90 is contained in the rotary porous polymer film container 3. The rotary porous polymer film container 3 includes one or more medium flow inlets. In one embodiment, the rotary porous polymer film container 3 includes a bottom part 30, a side part 31 disposed generally perpendicularly on the bottom part 30, and an apex part 32 disposed on the upper part of the side part 31, and forms a vessel having an appearance having a cylindrical shape. In another embodiment, the rotary porous polymer film container 3 may be, for example, a triangular prism, a quadrangular prism, a pentagonal prism, or a polygonal column. The bottom part 30 includes one or more first medium flow inlets 300. The side part 31 includes one or more second medium flow inlets 310. The apex part 32 includes one or more third medium flow inlets 320. The shapes of the first medium flow inlets 300, the second medium flow inlets 310, or the third medium flow inlets 320 may be, for example, circular, elliptical, triangular, quadrangular, pentagonal, hexagonal, polygonal, or the like. The sizes of the first medium flow inlets 300, the second medium flow inlets 310, and the third medium flow inlets 320 may be to such a degree that the modularized porous polymer film 90 does not protrude from the rotary porous polymer film container 3, and can be changed, if appropriate. The number of the first medium flow inlets 300, the second medium flow inlets 310, or the third medium flow inlets 320 is, for example, one, two, three, four, five, six, seven, eight, ten, twelve, fourteen, sixteen, eighteen, twenty, fifty, one hundred, or the like.

A medium is supplied/exhausted to the interior and exterior of the rotary porous polymer film container 3 through the first medium flow inlets 300, the second medium flow inlets 310, and the third medium flow inlets 320. In another embodiment, for example, the rotary porous polymer film container 3 may have a spherical shape, a conical shape, a pyramidal shape, frusto-conical shape, or a revolution solid shape of arbitrary polygon, or the like.

In the embodiment, the apex part 32 is detachable from and attachable to the side part 31, and the modularized porous polymer film 90 can be contained into and/or taken from the interior of the rotary porous polymer film container 3.

In one embodiment, the rotary porous polymer film container 3 includes a rotating part 33. In one embodiment, the rotating part 33 is disposed on the under surface of the bottom part 30 of the rotary porous polymer film container 3, and includes a rotational side part 330 having a cylindrical shape, a rotational bottom part 331, and rotative power reception means 333 fixed to the rotational bottom part 331. The rotative power reception means 333 is, for example, a magnet or a rotation shaft. In one embodiment, for example, a magnetic stirring bar may be used as the rotative power reception means 333. In one embodiment, when the rotative power reception means 333 is a magnet, for example, a magnetic stirrer can be used as rotational driving means 4, and can rotate the rotary porous polymer film container 3 in a non-contact manner. In another embodiment, when the rotative power reception means 333 is a rotation shaft, for example, a rotary motor connected to the rotation shaft can be used as the rotational driving means 4. The rotational motion of the rotational driving means 4 is transmitted to the rotative power reception means 333. As a result, the rotary porous polymer film container 3 is rotated. In the rotary porous polymer film container 3 according to the embodiment of FIGS. 2 to 4, the bottom face of the rotational bottom part 331 is rotated in contact with a culture vessel bottom part 22. In the case of the cell culture device 1 of the present invention, the rotary porous polymer film container 3 is rotated independently of the culture vessel 2, whereby oxygen and a nutrient can be sufficiently supplied to the modularized porous polymer film 90 to which cells are applied, and a large amount of cells can be cultured.

In one embodiment, the rotary porous polymer film container 3 in which the modularized porous polymer film 90 is contained can be instantly used as the cell culture device 1 by being combined with the culture vessel 2. The rotary porous polymer film container 3 can be moved to the culture vessel 2 filled with a fresh medium, and also easily enables medium replacement. Another embodiment may also be provided as a cell culture kit including the culture vessel 2 and the rotary porous polymer film container 3 containing the modularized porous polymer film 90.

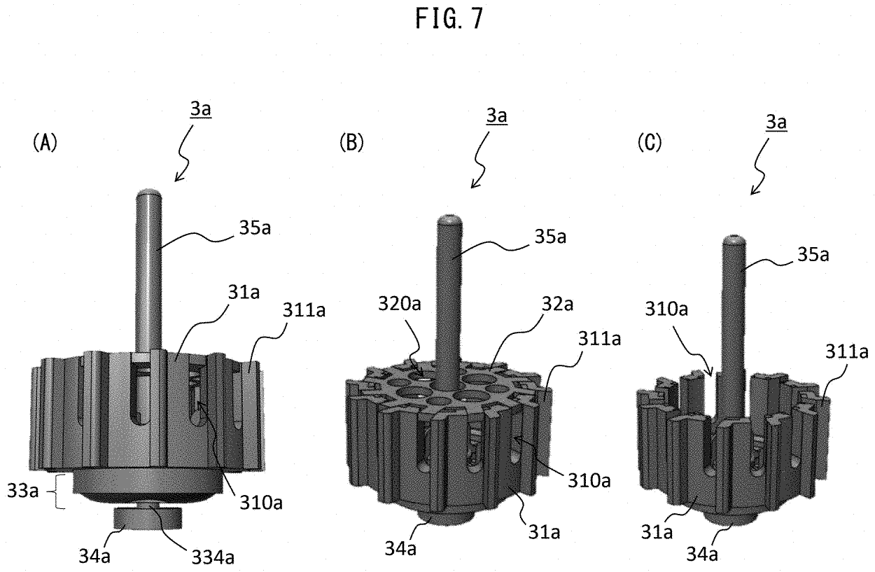

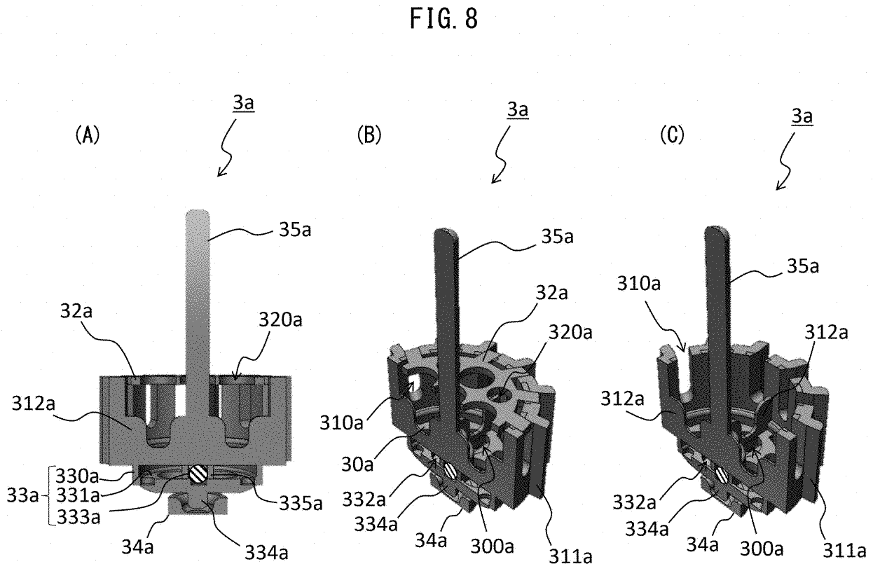

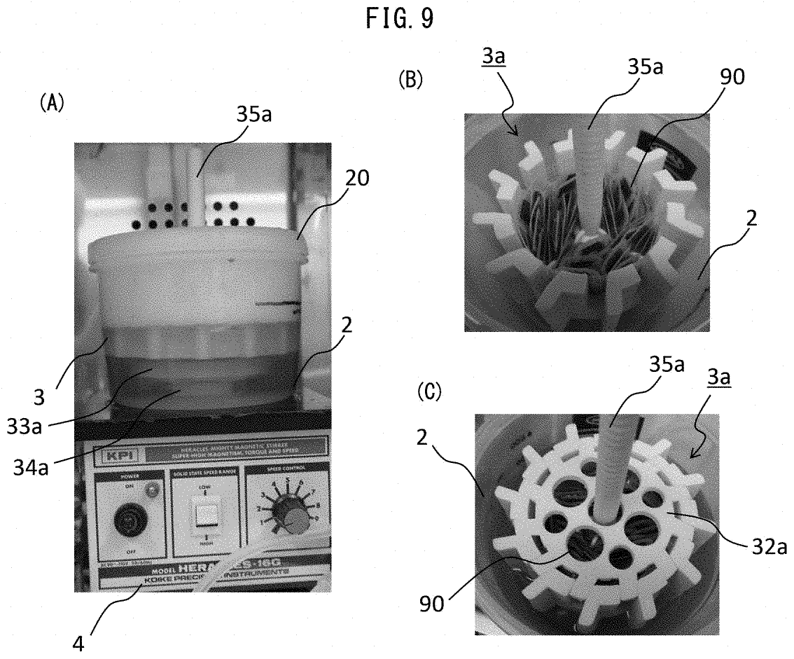

FIGS. 7 and 8 are diagrams illustrating a rotary porous polymer film container 3a applied to the cell culture device 1 in another embodiment, and FIG. 9 is a photograph illustrating the usage state thereof. The fundamental configuration and inventive concept of the rotary porous polymer film container 3a illustrated in FIGS. 7 and 8 are the same as those of each member of the rotary porous polymer film container 3. The above description of each corresponding member of the rotary porous polymer film container 3 is applied to the description of each member disposed in the rotary porous polymer film container 3a, denoted by the same reference numeral as that of each member disposed in the rotary porous polymer film container 3, except that “a” is added to the reference numeral of each member. Only members to which the description of each member of the rotary porous polymer film container 3 is not applied will now be described.

A shaft 35a for stabilizing the rotation of the rotary porous polymer film container 3a is disposed in the rotary porous polymer film container 3a. The shaft 35a penetrates a through-hole opened in a lid 20 (see FIG. 9). In another embodiment, the shaft 35a may be one that does not penetrate the lid 20 but is supported by a bearing disposed in the inside of the lid 20.

Rotating wings 311a may be disposed on a side part 31a. When the rotary porous polymer film container 3a is rotated, the rotating wings 311a allow liquid flow to occur, and oxygen in a gas phase can be efficiently taken into a medium. The shape and number of the rotating wings 311a can be adjusted as appropriate depending on the purpose thereof.

A rotation shaft member 334a is disposed on the lower part of a rotating part 33a. A bearing 34a for receiving the rotation shaft member 334a is disposed on the bottom face (not illustrated) of a culture vessel 2. The rotation shaft member 334a is supported by the recess of the bearing 34a, whereby the rotation of the rotary porous polymer film container 3a can be stabilized.

FIG. 8 is a cross-sectional view of the rotary porous polymer film container 3a illustrated in FIG. 7. The rotating part 33a includes rotative power reception means 333a (for example, a bar-like magnetic stirring bar). The rotative power reception means 333a is fixed by a fixation member 335a surrounding the rotative power reception means 333a.

Partitioned members 312a may be disposed in the interior of the rotary porous polymer film container 3a. For example, the partitioned members 312a can alleviate the imbalance of the modularized porous polymer film 90 caused by liquid flow when a modularized porous polymer film 90 is placed as in the case of FIG. 9(B), and the rotary porous polymer film container 3a is rotated. Such shapes and number of the partitioned members 312a that the imbalance of the modularized porous polymer film 90 can be alleviated and liquid flow is caused are acceptable, and can be adjusted, if appropriate.

2-2. Embodiment of Porous Polymer Film Used in Cell Culture Device

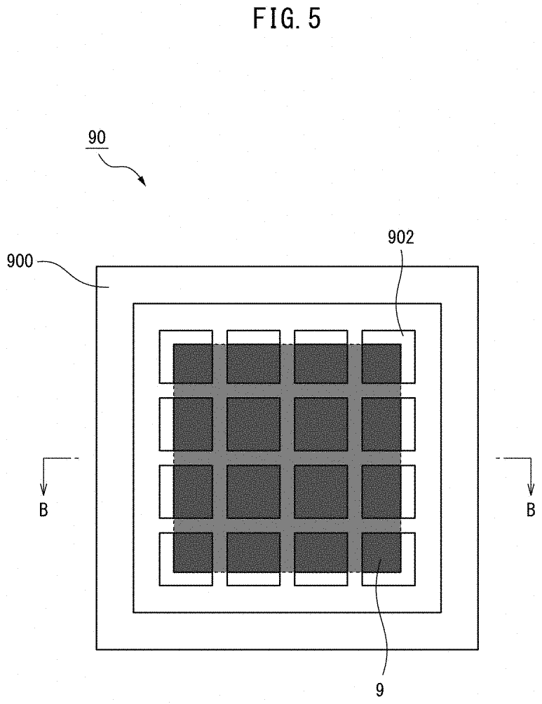

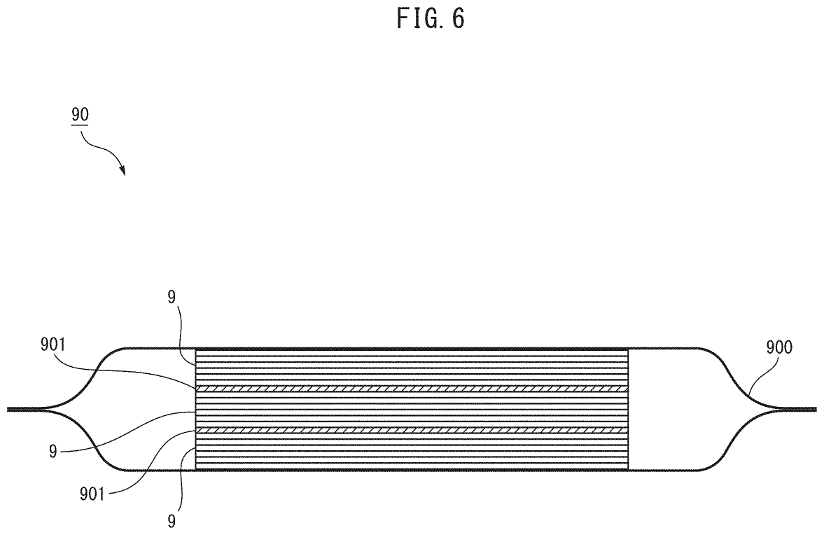

A modularized porous polymer film is used as a porous polymer film used in an embodiment of the present invention. In this specification, a “modularized porous polymer film” refers to a porous polymer film 9 contained in a casing 900 (for example, see FIGS. 5 and 6). In this specification, a “modularized porous polymer film” can be merely described as a “module”, and they mean the same thing even if being changed mutually.

In a modularized porous polymer film 90 used in the embodiment of the present invention,

(i) the two or more independent porous polymer films 9 being aggregated,

(ii) the porous polymer films 9 being folded up,

(iii) the porous polymer films 9 being wound into a roll-like shape, and/or

(iv) the porous polymer films 9 being tied into a rope-like shape

may be contained in the casing 900, and the modularized porous polymer film 90 can be applied to a rotary porous polymer film container 3.

In this specification, “the two or more independent porous polymer films 2 being aggregated are contained in a casing” refers to a state in which the two or more porous polymer films 9 that are independent of each other are aggregated and contained in a specific space surrounded by the casing 900. In the present invention, the two or more independent porous polymer films 9 may be ones in which at least one place of the porous polymer films 9 and at least one place in the casing 900 are fixed by an optional method, and the porous polymer films 9 are fixed so as to be in the state of being immovable in the casing 900. The two or more independent porous polymer films 9 may also be small pieces. The shape of the small pieces may be an optional shape such as, for example, a circular, elliptical, quadrangular, triangular, polygonal, or string shape, and is preferably a string or quadrangular shape. In the present invention, the size of the small pieces may be an optional size. When the small pieces have a string shape, the small pieces may have an optional length, and the preferred width of the small pieces is 80 mm or less, preferably 30 mm or less, and more preferably 10 mm or less. As a result, stress is prevented from being applied to cells growing in the porous polymer films 9. In the present invention, when the small pieces of the porous polymer films 9 have a quadrangular shape, the small pieces more preferably have an approximately square shape, and the length of one side thereof may be formed to be along the inner wall of the casing or to be shorter than the length of one side of the inner wall (for example, shorter by around 0.1 mm to 1 mm) so as to be in a state in which the porous polymer films are immovable in the casing 900. In the present invention, when the small pieces of the porous polymer films 9 have an approximately square shape, the length thereof may be an optional length, and, for example, the preferred length thereof is 80 mm or less, preferably 50 mm or less, more preferably 30 mm or less, and still more preferably 20 mm or less, and may be 10 mm or less.

In this specification, “porous polymer films being folded up” are the porous polymer films 9 that are allowed to be in the state of being immovable in the casing 900 due to friction against each surface of the porous polymer films 9 and/or a surface in the casing 900 by being folded up in the casing 900. In this specification, “being folded” may mean a state in which creases are or are not put in the porous polymer films 9.

In this specification, “porous polymer films being wound into a roll-like shape” refer to the porous polymer films 9 that are allowed to be in the state of being immovable in the casing 900 due to friction against each surface of the porous polymer films 9 and/or a surface in the casing 900 by winding the porous polymer films 9 into a roll-like shape. In the present invention, the porous polymer films 9 being woven into a rope-like shape refer to the porous polymer films 9 that are allowed to be in the state of being immovable due to friction between the porous polymer films 9 by, for example, weaving the plural porous polymer films 9 having a strip shape into a rope-like shape by an optional method. It is also acceptable to combine (i) the porous polymer films 9 in which the two or more independent porous polymer films 9 are aggregated, (ii) the porous polymer films 9 that are folded up, (iii) the porous polymer films 9 that are wound into a roll-like shape, and (iv) the porous polymer films 9 that are tied into a rope-like shape, and they may be contained in the casing 900.

In this specification, a “state in which the porous polymer film is immovable in a casing” refers to a state in which the porous polymer films 9 are contained in the casing 900 so as to be in the state of having a shape prevented from being continuously changed when the modularized porous polymer film 90 is cultured in a cell culture medium. In other words, the state is a state in which the porous polymer films 9 themselves are inhibited from continuously waving due to fluid. Since the porous polymer films 9 keep being in the state of being immovable in the casing 900, stress is prevented from being applied to cells growing in the porous polymer films 9, and the cells can be stably cultured without killing the cells.

FIGS. 5 and 6 illustrate the structure of the modularized porous polymer film 90 according to one embodiment. In the modularized porous polymer film 90, a layered body of the plural porous polymer films 9 is contained in the casing 900. The layered porous polymer films 9 may be small pieces. The shape of the small pieces may be an optional shape such as, for example, a circular, elliptical, quadrangular, triangular, polygonal, or string shape. Preferably, the small pieces of the layered porous polymer films 9 have an approximately square shape. The size of the small pieces may be an optional size. When the small pieces have an approximately square shape, the length thereof is not particularly limited but is, for example, 80 mm or less, 50 mm or less, 30 mm or less, 20 mm or less, or 10 mm or less.

When the porous polymer films 9 contained in the casing 900 are a layered body of the plural porous polymer films 9, the layered body is preferably a layered body of two or more, three or more, four or more, or five or more, and 100 or less, 50 or less, 40 or less, 30 or less, 20 or less, 15 or less, or 10 or less porous polymer films, more preferably a layered body of 3 to 100 porous polymer films, and more preferably a layered body of 5 to 50 porous polymer films.

When the porous polymer films 9 contained in the casing 900 are a layered body of the plural porous polymer films, liners 901 may be disposed between the porous polymer films 9 (see FIG. 6). A medium can be efficiently supplied to between the layered porous polymer films 9 by disposing the liners 901. The liners 901 are not particularly limited as long as having the function of forming optional spaces between the layered porous polymer films 9 and efficiently supplying a medium. For example, a planar structure having a mesh structure can be used as such a liner 901. For example, a mesh made of polystyrene, polycarbonate, polymethyl methacrylate, polyethylene terephthalate, or stainless steel can be used as the material of the liners 901. However, the material is not limited thereto. When the liners 901 having a mesh structure is possessed, openings to such a degree that a medium can be supplied to between the layered porous polymer films 9 may be included, and can be selected if appropriate.

3. Method of Culturing Cells Using Cell Culture Device

In this specification, a “medium” refers to a cell culture medium for culturing a cell, particularly an animal cell. The medium is used as a meaning synonymous with a cell culture medium. Therefore, the medium used in the present invention refers to a liquid medium. A medium that is normally used can be used as the kind of the medium, which is determined depending on the kind of a cell to be cultured, if appropriate.

<Step of Applying Cells to Porous Polymer Film>

A specific step of applying cells to a porous polymer film, used in the present invention, is not particularly limited. The step described herein or an optional technique suitable for applying a cell to a film-like carrier can be adopted. Without limitation, in the method of the present invention, the application of a cell to the porous polymer film includes, for example, the following modes.

(A) A mode including a step of seeding cells on a surface of the porous polymer film;

(B) a mode including a step of

putting a cell suspension on a dry surface of the porous polymer film,

leaving it standing, moving the porous polymer film to promote the flow of the liquid, or stimulating a part of the surface to allow the film to absorb the cell suspension, and

allowing the cells to remain in the cell suspension in the film, and allowing water to flow out; and

(C) a mode including a step of

moisturizing one surface or both surfaces of the porous polymer film with a cell culture medium or a sterilized liquid,

loading a cell suspension on the moisturized porous polymer film, and

allowing the cells to remain in the cell suspension in the film, and allowing water to flow out.

The mode (A) includes directly seeding cells or a cell mass on the surface of the porous polymer film. Alternatively, a mode in which a porous polymer film is put in a cell suspension, and a cell culture medium is infiltrated from the surface of the film is also included.

The cells seeded on the surface of the porous polymer film adhere to the porous polymer film and moves into pores. Preferably, the cells adhere to the porous polymer film even if a physical or chemical force is not particularly applied from the exterior. The cells seeded on the surface of the porous polymer film can stably grow and proliferate on the surface and/or in the interior of the film. The various different shapes of the cells can be formed depending on the position of the film, at which the cells grow and proliferate.

In the mode (B), the cell suspension is put on the dry surface of the porous polymer film. The cell suspension is infiltrated into the film by leaving the porous polymer film standing, moving the porous polymer film to promote the flow of the liquid, or stimulating a part of the surface to allow the film to absorb the cell suspension. Without wishing to be bound by theory, it is considered that this depends on a property derived from each surface shape of the porous polymer film. In accordance with this mode, the cells are absorbed and seeded in the place of the film, at which the cell suspension is loaded.

Alternatively, like the mode (C), it is also acceptable to moisturize a part or the whole of one surface or both surfaces of the porous polymer film with a cell culture medium or a sterilized liquid, and then loading a cell suspension on the moisturized porous polymer film. In this case, the passage rate of the cell suspension is greatly improved.

For example, a method in which a part of a film pole is moisturized (hereinafter, referred to as a “one-point wet method”) can be used for the main purpose of preventing scatter of a film. The one-point wet method is almost near to a dry method in which a film is not substantially moisturized (the mode (B)). However, membrane permeation of cell-containing fluid is considered to be rapid in a small moisturized portion. A method in which a cell suspension is loaded on the sufficiently moisturized whole of one surface or both surfaces of the porous polymer film (hereinafter, referred to as a “wet film”) can also be used (hereinafter, referred to as a “wet film method”). In this case, the passage rate of the cell suspension is greatly improved in the whole of the porous polymer film.

In the modes (B) and (C), the cells in the cell suspension are allowed to remain in the film, and water is allowed to flow out. As a result, treatment such as an increase in the concentration of the cells in the cell suspension, or flow-out of an unnecessary component other than the cells, together with water, is also enabled.

The mode (A) may be referred to as “natural seeding”, and the modes (B) and (C) may be referred to as “absorption seeding”.

Without limitation, preferably, viable cells selectively remain in the porous polymer film. Thus, in a preferred embodiment of the method of the present invention, viable cells remain in the porous polymer film, and dead cells preferentially flow out together with water.

The sterilized liquid used in the mode (C) is not particularly limited, but is a sterilized buffer or sterilized water. The buffer is, for example, (+) and (−) Dulbecco's PBS, (+) and (−) Hank's balanced salt solution, or the like. Examples of the buffer are set forth in the following Table 1.

TABLE 1

| Component | Concentration (mmol/L) | Concentration (g/L) |
|---|---|---|
| NaCl | 137 | 8.00 |
| KCl | 2.7 | 0.20 |
| Na₂HPO₄ | 10 | 1.44 |
| KH₂PO₄ | 1.76 | 0.24 |
| pH (−) | 7.4 | 7.4 |

Further, in the method of the present invention, the application of cells to the porous polymer films also includes a mode in which adherent cells in a floating state are allowed to coexist with the porous polymer films in a suspension manner, to thereby allow the cells to adhere to the films (entanglement). For example, in the method of the present invention, a cell culture medium, cells, and the one or more porous polymer films may be put in a cell culture vessel in order to apply the cells to the porous polymer films. When the cell culture medium is liquid, the porous polymer films exist in the state of floating in cell culture medium. The cells can adhere to the porous polymer films based on the properties of the porous polymer films. Thus, even in the case of adherent cells that are not suitable for float culture by nature, use of the porous polymer films enables the cells to be cultured while floating the cells in a state in which the cells are adsorbed in the porous polymer films. Preferably, the cells adhere to the porous polymer films. “Voluntarily adhere” means that cells remain on the surfaces or in the interiors of the porous polymer films even if a physical or chemical force is not particularly applied from the exterior.

For the above-described application of the cells to the porous polymer films, two or more methods may be used in combination. For example, the cells may be applied to the porous polymer films in combination of two or more methods of the modes (A) to (C). It is possible to apply the porous polymer films, on which the cells are carried, to a porous polymer film mounting unit in the cell culture device described above, and to culture the cells.

In addition, it is also acceptable to dropwise add a medium containing suspended cells to a porous polymer film container in which a modularized porous polymer film is contained, and to seed the cells.

Alternatively, cells can be homogeneously seeded on the modularized porous polymer film by filling a cell suspension containing the cells into the culture vessel 2 (for example, filling a cell suspension to a cell suspension level L). As a result, the cells seeded on the porous polymer films are prevented from being partly confluent, and can efficiently grow.

In this specification, examples of “suspended cells” include cells obtained by allowing adherent cells to forcedly float due to a proteolytic enzyme such as trypsin and by suspending the cells in a medium, and adherent cells enabled to be subjected to float culture in the medium by a known conditioning step.

The types of the cells which may be used for the present invention may be selected from the group consisting of animal cells, insect cells, plant cells, yeast cells and bacteria. Animal cells are largely divided into cells from animals belonging to the subphylum Vertebrata, and cells from non-vertebrates (animals other than animals belonging to the subphylum Vertebrata). There are no particular restrictions on the source of the animal cells, for the purpose of the present specification. Preferably, they are cells from an animal belonging to the subphylum Vertebrata. The subphylum Vertebrata includes the superclass Agnatha and the superclass Gnathostomata, the superclass Gnathostomata including the class Mammalia, the class Aves, the class Amphibia and the class Reptilia. Preferably, they are cells from an animal belonging to the class Mammalia, generally known as mammals. Mammals are not particularly restricted but include, preferably, mice, rats, humans, monkeys, pigs, dogs, sheep and goats.

The types of animal cells or plant cells that may be used for the invention are not particularly restricted, but are preferably selected from the group consisting of pluripotent stem cells, tissue stem cells, somatic cells and germ cells.

The term “pluripotent stem cells”, in this specification, is intended as a comprehensive term for stem cells having the ability to differentiate into cells of any tissues (pluripotent differentiating power). While not restrictive, pluripotent stem cells include embryonic stem cells (ES cells), induced pluripotent stem cells (iPS cells), embryonic germ cells (EG cells) and germ stem cells (GS cells). They are preferably ES cells or iPS cells. Particularly preferred are iPS cells, which are free of ethical problems, for example. The pluripotent stem cells used may be any publicly known ones, and for example, the pluripotent stem cells described in WO2009/123349 (PCT/JP2009/057041) may be used.

The term “tissue stem cells” refers to stem cells that are cell lines capable of differentiation but only to limited specific tissues, though having the ability to differentiate into a variety of cell types (pluripotent differentiating power). For example, hematopoietic stem cells in the bone marrow are the source of blood cells, while neural stem cells differentiate into neurons. Additional types include hepatic stem cells from which the liver is formed and skin stem cells that form skin tissue. Preferably, the tissue stem cells are selected from among mesenchymal stem cells, hepatic stem cells, pancreatic stem cells, neural stem cells, skin stem cells and hematopoietic stem cells.

The term “somatic cells” refers to cells other than germ cells, among the cells composing a multicellular organism. In sexual reproduction, these are not passed on to the next generation. Preferably, the somatic cells are selected from among hepatocytes, pancreatic cells, muscle cells, bone cells, osteoblasts, osteoclasts, chondrocytes, adipocytes, skin cells, fibroblasts, pancreatic cells, renal cells and lung cells, or blood cells such as lymphocytes, erythrocytes, leukocytes, monocytes, macrophages or megakaryocytes.

The term “germ cells” refers to cells having the role of passing on genetic information to the succeeding generation in reproduction. These include, for example, gametes for sexual reproduction, i.e. the ova, egg cells, sperm, sperm cells, and spores for asexual reproduction.

The cells may also be selected from the group consisting of sarcoma cells, established cell lines and transformants. The term “sarcoma” refers to cancer occurring in non-epithelial cell-derived connective tissue cells, such as the bone, cartilage, fat, muscle or blood, and includes soft tissue sarcomas, malignant bone tumors and the like. Sarcoma cells are cells derived from sarcoma. The term “established cell line” refers to cultured cells that are maintained in vitro for long periods and reach a stabilized character and can be semi-permanently subcultured. Cell lines derived from various tissues of various species including humans exist, such as PC12 cells (from rat adrenal medulla), CHO cells (from Chinese hamster ovary), HEK293 cells (from human embryonic kidney), HL-60 cells (from human leukocytes) and HeLa cells (from human cervical cancer), Vero cells (from African green monkey kidney epithelial cells), MDCK cells (from canine renal tubular epithelial cells), HepG2 cells (from human hepatic cancer), BHK cells (new-born hamster kidney cell), NIH3T3 cells (from mouse fetal fibroblast cells). The term “transformants” refers to cells with an altered genetic nature by extracellularly introduced nucleic acid (DNA and the like).

In this specification, an “adherent cell” is generally a cell which is required to adhere itself on an appropriate surface for growth, and is also referred to as an adhesion cell or an anchorage-dependent cell. In certain embodiments of the present invention, the cells used are adherent cells. The cells used for the present invention are adherent cells, more preferably cells which may be cultured even as a suspension in a medium. The adherent cells which can be suspension cultured may be obtained by conditioning the adherent cells to a state suitable for suspension culture, and include, for example, CHO cells, HEK293 cells, Vero cells, NIH3T3 cells, and cell lines derived from these cells.

In the cell culture method of the invention, application of cells and culturing are carried out on a porous polymer film, thereby allowing simple culture of large volumes of cells to be accomplished since large numbers of cells grow on the multisided connected pore sections on the inside, and the surfaces on the porous polymer film. It is possible to provide an environment in which the cells used in the present invention and seeded on the porous polymer film can grow even under a stirring condition under which conventional cells are killed, and to culture a large amount of the cells.

Throughout the present specification, the volume of the porous polymer film without cells, that occupies the space including the volume between the interior gaps, will be referred to as the “apparent porous polymer film volume”. In the state where the cells are applied to the porous polymer film and the cells have been supported on the surface and the interior of the porous polymer film, the total volume of the porous polymer film, the cells and the medium that has wetted the porous polymer film interior, which is occupying the space therein, will be referred to as the “porous polymer film volume including the cell survival zone”. When the porous polymer film has a film thickness of 25 μm, the porous polymer film volume including the cell survival zone is a value of at maximum about 50% larger than the apparent porous polymer film volume. In the method of the invention, a plurality of porous polymer films may be housed in a single cell culture vessel for culturing, in which case the total sum of the porous polymer film volume including the cell survival zone for each of the plurality of porous polymer films supporting the cells may be referred to simply as the “total sum of the porous polymer film volume including the cell survival zone”.

Using the method of the invention, cells can be satisfactorily cultured for a long period of time even under conditions in which the total volume of the cell culture medium in the cell culture vessel is 10,000 times or less of the total sum of the porous polymer film volume including the cell survival zone. Moreover, cells can be satisfactorily cultured for a long period of time even under conditions in which the total volume of the cell culture medium in the cell culture vessel is 1,000 times or less of the total sum of the porous polymer film volume including the cell survival zone. In addition, cells can be satisfactorily cultured for a long period of time even under conditions in which the total volume of the cell culture medium in the cell culture vessel is 100 times or less of the total sum of the porous polymer film volume including the cell survival zone. In addition, cells can be satisfactorily cultured for a long period of time even under conditions in which the total volume of the cell culture medium in the cell culture vessel is 10 times or less of the total sum of the porous polymer film volume including the cell survival zone.

In other words, according to the invention, the space (vessel) used for cell culturing can be reduced to an absolute minimum, compared to a conventional cell culture device for performing two-dimensional culture. Furthermore, when it is desired to increase the number of cells cultured, the cell culturing volume can be flexibly increased by a convenient procedure including increasing the number of layered porous polymer films. In a cell culture device comprising a porous polymer film to be used for the invention, the space (vessel) in which cells are cultured and the space (vessel) in which the cell culture medium is stored can be separate, and the necessary amount of cell culture medium can be prepared according to the number of cells to be cultured. The space (vessel) in which the cell culture medium is stored can be increased or decreased according to the purpose, or it may be a replaceable vessel, with no particular restrictions.

In this specification, “mass culturing of cells” refers to culturing in which the number of cells in the cell culture vessel after culturing using the porous polymer film reaches 1.0×10⁵ or more, 1.0×10⁶ or more, 2.0×10⁶ or more, 5.0×10⁶ or more, 1.0×10⁷ or more, 2.0×10⁷ or more, 5.0×10⁷ or more, 1.0×10⁸ or more, 2.0×10⁸ or more, 5.0×10⁸ or more, 1.0×10⁹ or more, 2.0×10⁹ or more, or 5.0×10⁹ or more per milliliter of medium, assuming that all of the cells are evenly dispersed in the cell culture medium in the cell culture vessel.

It should be noted that as a method for measuring cell count during or after culture, various known methods may be used. For example, as the method for counting the number of cells in the cell culture vessel after culturing using the porous polymer film, assuming that the cells are evenly dispersed in the cell culture medium in the cell culture vessel, any publicly known method may be used. For example, a cell count method using CCK8 (see the following sentence) may be suitably used. Specifically, a Cell Counting Kit 8 (a solution reagent, commercially available from Dojindo Laboratories)(hereunder referred to as “CCK8”) may be used to count the number of cells in ordinary culturing without using a porous polymer film, and the correlation coefficient between the absorbance and the actual cell count is determined. Subsequently, the cells are applied, the cultured porous polymer film may be transferred to CCK8-containing medium and stored in an incubator for 1 to 3 hours, and then the supernatant is extracted and its absorbance is measured at a wavelength of 460 nm, and the cell count is determined from the previously calculated correlation coefficient.

In addition, from another point of view, for example, “mass culturing of cells” may refer to culturing in which the number of cells in the cell culture vessel after culturing using the porous polyimide film reaches 1.0×10⁵ or more, 2.0×10⁵ or more, 1.0×10⁶ or more, 2.0×10⁶ or more, 5.0×10⁶ or more, 1.0×10⁷ or more, 2.0×10⁷ or more or 5.0×10⁷ or more, 1.0×10⁸ or more, 2.0×10⁸ or more, or 5.0×10⁸ or more, per square centimeter of porous polymer film. The number of cells contained per square centimeter of porous polymer film may be appropriately measured using a publicly known method, such as with the CCK8 described above.

### EXAMPLES

The present invention will now be explained in greater detail by Examples. It is to be understood, however, that the invention is not limited to these Examples. A person skilled in the art may easily implement modifications and changes to the invention based on the description in the present specification, and these are also encompassed within the technical scope of the invention.

The porous polyimide films used in the following examples were prepared by forming a polyamic acid solution composition including a polyamic acid solution obtained from 3,3′,4,4′-biphenyltetracarboxylic dianhydride (s-BPDA) as a tetracarboxylic acid component and 4,4′-diaminodiphenyl ether (ODA) as a diamine component, and polyacrylamide as a coloring precursor, and performing heat treatment at 250° C. or higher. The resulting porous polyimide film was a three-layer structure porous polyimide film having a surface layer A and a surface layer B, the surface layers having a plurality of pores, and a macrovoid layer sandwiched between the surface layers A and B; wherein the average pore diameter of the pore present on the surface layer A was 19 μm, the average pore diameter of the pore present on the surface layer B was 42 μm, and the film thickness was 25 μm, and the porosity was 74%.

### Example 1

Conditioned/suspended anti-human IL-8 antibody producing CHO-DP12 cells (ATCC CRL-12445) were float-cultured using a medium (BalanCD (Trademark) CHO Growth A) and culture was continued until viable cell count per mL was 3.51×10⁶ cells/mL (total cell number of 3.83×10⁶ cells/mL, viable cell rate of 92%). One hundred modules having the structure illustrated in FIGS. 5 and 6 were prepared, washed with diluted Milton (registered trademark) (KYORIN Pharmaceutical Co., Ltd., Japan), ultrapure water, and 70% ethanol-containing water, and then dried in a sterilization manner, followed by completing the preparation. Variations with time are set forth in Table 2. The size of the porous polyimide films used in the modules is 1.0×1.0 cm, the total number of the porous polyimide films are 1800, and the total area thereof is 1800 cm². A rotary porous polymer film container prepared by a 3D printer (FIGS. 2 to 4), the 100 modules in total were placed in the upper cavity portion thereof in a sterilization manner, and the rotary porous polymer film container was placed in the interior of a transparent container. A magnetic stirrer was placed in the lower portion of a bioreactor, the bioreactor was then transported into a CO₂ incubator, and the preparation of culture were completed. The upper open portion of the transparent container was covered with a non-woven fabric, and the transparent container was allowed to be in a form in which air was able to be taken in while avoiding contamination.

To the rotation culture device prepared as described above, 200.0 mL of CHO cell monolayer culture medium KBM270 manufactured by Kohjin Bio Co., Ltd. was added, and the modules was immersed in the medium for 10 minutes at a rotation speed of 56 rpm. A liquid mixture of 30.8 mL of CHO DP-12 floating cell culture medium (total cell number of 3.83×10⁶ cells/mL, viable cell count of 3.51×10⁶ cells/mL, dead cell count of 3.23×10⁵ cells/mL, and viable cell rate of 92%) and 69.2 mL of a medium for floating cells (BalanCD (trade mark) CHO Growth A) was added thereto, the resultant was gently stirred and mixed for 5 minutes in a CO₂ incubator, and then left standing for 2.5 hours, and cell adsorption was performed. Almost no cells were observed in the medium collected after 2.5 hours, and the cell adsorption ratio was 100%. The expected average cell density at this time point was 6.00×10⁴ cells/cm². The medium used in the cell adsorption was discharged, 200 mL of CHO cell monolayer culture medium KBM270 manufactured by Kohjin Bio Co., Ltd. was poured into the rotational bioreactor vessel, and the bioreactor was rotated at a rotation speed of 56 rpm to continue the culture. After 3 days, the rotation speed was increased to 192 rpm, and the culture was allowed to proceed. Medium replacement was performed every day, and the amounts of consumed glycol, produced lactic acid, lactate dehydrogenase, and produced antibody per day in the medium were measured for 4 days using Cedex Bio manufactured by Roche Diagnostics K.K. It was observed that the consumption of glucose and the production of lactic acid were improved with time, and the stable cell culture proceeded. The results are set forth in Table 2.

TABLE 2

| | Amount of | Amount of Produced |
|---|---|---|
| Day | Consumed Glucose mg/L | Lactic Acid mg/L |
| Day 1 | 236 | 272 |
| Day 2 | 584 | 446 |
| Day 3 | 830 | 680 |
| Day 4 | 647 | 459 |

### Example 2

Conditioned/suspended anti-human IL-8 antibody producing CHO-DP12 cells (ATCC CRL-12445) were float-cultured using a medium (BalanCD (Trademark) CHO Growth A) and culture was continued until viable cell count per mL was 2.29×10⁶ cells/mL (total cell number of 2.62×10⁶ cells/mL, viable cell rate of 88%).

One hundred modules having the structure illustrated in FIGS. 5 and 6 were put in a bag for the sterilization (manufactured by ThermoFisher Scientific Inc.), the bag was subjected to sterilization by irradiation with gamma rays with a minimum of 25 kGy and a maximum of 50 kGy, and the preparation of the modules was completed. The size of the porous polyimide films used in the modules was 1.0 cm×1.0 cm, the total number of the porous polyimide films was 1,800, and the total area thereof was 1,800 cm².

A rotary porous polymer film container (FIGS. 7 to 9) that contained the modules and rotated was produced by a 3D printer, and then sterilized by a method similar to that in the case of the modules.

In order to enable aeration into the liquid in the container, a sparger made of glass was placed in the lower part of the transparent container in which the above-described rotary porous polymer film container was placed. A bearing (see FIGS. 7 to 8) was placed in the center of the transparent container using an adhesive, and sufficiently left standing until the bearing was fixed. Like the modules, the bearing was sterilized by the gamma-ray method in this state.

One hundred modules in total were placed in the module container of the rotation culture device in a sterilization manner (see FIG. 9 (B)), and the module container was placed on the central bearing in the transparent container. A magnetic stirrer was placed in a CO₂ incubator, and the rotation culture device was placed thereon.

Into the bioreactor prepared as described above, 450 mL of a medium for culturing a CHO cell monolayer KBM CHO HBM1 (manufactured by Kohjin Bio Co., Ltd.) was added, and the modules were immersed in the medium for about 30 minutes at a rotation speed of 60 rpm. The medium for culturing a CHO cell monolayer KBM CHO HBM1 (manufactured by Kohjin Bio Co., Ltd.) was discarded in an amount of 50 mL, 50 mL of a suspension containing CHO DP-12 (total cell number of 2.62×10⁶ cells/mL, viable cell count of 2.29×10⁶ cells/mL, dead cell count of 3.23×10⁵ cells/mL, and viable cell rate of 88%) was added and left standing for about 1.5 hours, the culture was then performed for about 18.5 hours in a state in which the bioreactor was rotated at a rotation speed of 60 rpm, and the cells were allowed to be adsorbed in the modules for about 20 hours in total (expected average viable cell adsorption number of 6.36×10⁴ cells per sheet). The viable cell adsorption ratio calculated from the collected medium was 94%.

Then, the medium was removed, 450 mL of a medium for culturing a CHO cell monolayer KBM CHO HBM1 (manufactured by Kohjin Bio Co., Ltd.) was added, and the culture was performed while performing aeration at an oxygen concentration of 40% at such a flow rate that bubbles do not overflow from the vessel. Medium replacement was performed 4 and 10 days after the start of the culture. Sampling in a small amount was performed every day, and the amounts of consumed glucose, produced lactic acid, lactate dehydrogenase, and produced antibody per day in the medium were measured using Cedex Bio (manufactured by Roche Diagnostics K.K.). It was confirmed that glucose was consumed with time, and an antibody and lactic acid were continuously produced.

The concentrations of consumed glucose and the concentrations of produced lactic acid for 4 days from the start of the culture to initial medium replacement are set forth in Table 3.

TABLE 3

| Culture | Concentration of | Concentration of Produced |
|---|---|---|
| Day | Consumed Glucose mg/L/day | Lactic Acid mg/L/day |
| Day 1 | 817 | 367 |
| Day 2 | 357 | 412 |
| Day 3 | 474 | 419 |
| Dav 4 | 343 | 377 |

### Example 3

Human skin fibroblasts (Lonza CAT #CC-2511) were cultured in a medium (KBM Fibro Assist) manufactured by Kohjin Bio Co., Ltd. using 14 petri dishes of 150 cm² manufactured by AS ONE Corporation until achieving 6,500 cells/cm².

Thirty modules having the structure illustrated in FIGS. 5 and 6 were put in a bag for the sterilization (manufactured by ThermoFisher Scientific Inc.), and the bag was subjected to sterilization by irradiation with gamma rays. The size of the porous polyimide films used in the modules was 1.0 cm×1.0 cm, the total number of the porous polyimide films was 540, and the total area thereof was 540 cm². A rotational reactor having a shape similar to that in Example 2 prepared by the 3D printer, and a transparent container to which a bearing for placing the reactor was applied were prepared. Unlike Example 2, sparger made of glass was not placed, and a system of supplying oxygen only by rotation was adopted, in the present example. Like the modules, a rotation culture vessel and a transparent container were sterilized by a gamma-ray method, the 30 modules in total were then placed in the module container of the rotational reactor in a sterilization manner, and the module container was placed in the transparent container.

A magnetic stirrer was placed in a CO₂ incubator, and a rotation culture device was placed thereon.

Into the bioreactor prepared as described above, 270 mL of a medium (KBM Fibro Assist) manufactured by Kohjin Bio Co., Ltd. was added, and the modules were immersed in the medium for about 30 minutes at a rotation speed of about 60 rpm.

Thirty milliliters of a suspension containing human skin fibroblasts (total cell number of 1.23×10⁶ cells/mL, viable cell count of 1.00×10⁶ cells/mL, dead cell count of 2.30×10⁵ cells/mL, and viable cell rate of 81%) was added and left standing for about 1 hour, the culture was then performed for about 23 hours in a state in which the bioreactor was rotated at a rotation speed of about 60 rpm, and the cells were allowed to be adsorbed in the modules for about 24 hours in total (expected average viable cell adsorption number of 5.56×10⁴ cells per sheet). The viable cell adsorption ratio calculated from the medium after about 5 hours of the start of the adsorption was 95%.

Then, the medium was removed, 300 mL of a medium (KBM Fibro Assist) manufactured by Kohjin Bio Co., Ltd. was added, and the culture was performed. Medium replacement was performed basically at intervals of three or four days after the start of the culture, and the amounts of consumed glucose, produced lactic acid, and lactate dehydrogenase in the medium were measured using Cedex Bio (manufactured by Roche Diagnostics K.K.). It was confirmed that glucose was consumed with time, and lactic acid was continuously produced.

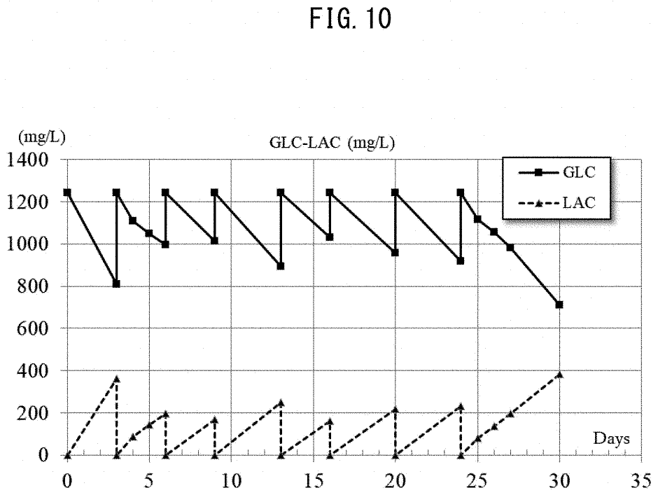

The concentrations of glucose and lactic acid after the start of the culture are illustrated in FIG. 10.

### REFERENCE SIGNS LIST

1 Cell culture device
2 Culture vessel
20 Lid
21 Filter
22 Culture vessel bottom part
3, 3a Rotary porous polymer film container
30, 30a Bottom part
300, 300a First medium flow inlet
31, 31a Side part
310, 310a Second medium flow inlet
311a Rotating wing
312a Partitioned member
32, 32a Apex part
320, 320a Third medium flow inlet
33, 33a Rotating part
330, 330a Rotational side part
331, 331a Rotational bottom part
332, 332a Fourth medium flow inlet
333, 333a Rotative power reception means
334a Rotation shaft member
335a Fixation member
34a Bearing
35a Shaft
4 Rotational driving means
9 Porous polymer film
90 Modularized porous polymer film
900 Casing
L Cell suspension level

1. A cell culture device comprising:
a culture vessel;
a rotary porous polymer film container that is contained in the culture vessel and has one or more medium flow inlets; and
a porous polymer film contained in the rotary porous polymer film container;
wherein the porous polymer film is a porous polymer film with a three-layer structure, comprising a surface layer A and a surface layer B having a plurality of pores, as well as a macrovoid layer sandwiched between the surface layer A and the surface layer B; an average pore diameter of the pores present in the surface layer A is smaller than an average pore diameter of the pores present in the surface layer B; the macrovoid layer comprises a partition wall bonded to surface layers A and B, and a plurality of macrovoids surrounded by the partition wall and the surface layers A and B; and the pores in the surface layers A and B communicate with the macrovoids;
wherein the porous polymer film is a modularized porous polymer film; and
wherein the rotary porous polymer film container is rotated independently of the culture vessel.

2. The cell culture device according to claim 1, further comprising rotational driving means for rotating the rotary porous polymer film container.

3. The cell culture device according to claim 2, wherein the rotary porous polymer film container comprises rotative power reception means.

4. The cell culture device according to claim 3, wherein the rotative power reception means is a magnet.

5. The cell culture device according to claim 4, wherein the rotational driving means is a magnetic stirrer.

6. The cell culture device according to any one of claims 1 to 5,
wherein the modularized porous polymer film is a modularized porous polymer film comprising a casing; and
wherein in the modularized porous polymer film,
(i) the two or more independent porous polymer films being aggregated,
(ii) the porous polymer films being folded up,
(iii) the porous polymer films being wound into a roll-like shape, and/or
(iv) the porous polymer films being tied into a rope-like shape are contained in the casing.

7. The cell culture device according to any one of claims 1 to 6, wherein the porous polymer film has a plurality of pores having an average pore diameter of 0.01 to 100 μm.

8. The cell culture device according to any one of claims 1 to 7, wherein an average pore diameter of the surface layer A is 0.01 to 50 μm.

9. The cell culture device according to any one of claims 1 to 8, wherein an average pore diameter of the surface layer B is 20 to 100 μm.

10. The cell culture device according to any one of claims 1 to 9, wherein a total film thickness of the porous polymer film is 5 to 500 μm.

11. The cell culture device according to any one of claims 1 to 10, wherein the porous polymer film is a porous polyimide film.

12. The cell culture device according to claim 11, wherein the porous polyimide film is a porous polyimide film comprising a polyimide derived from tetracarboxylic dianhydride and diamine.

13. The cell culture device according to claim 11 or 12, wherein the porous polyimide film is a colored porous polyimide film that is obtained by molding a polyamic acid solution composition comprising a polyamic acid solution derived from tetracarboxylic dianhydride and diamine, and a coloring precursor, and subsequently heat-treating the resultant composition at 250° C. or more.

14. The cell culture device according to any one of claims 1 to 10, wherein the porous polymer film is a porous polyethersulfone (PES) film.

15. A method of culturing cells, using the cell culture device according to any one of claims 1 to 14.
